(19) United States
(12) Patent Application Publication
(10) Pub. No.: US 2020/0312429 A1
(43) Pub. Date: Oct. 1, 2020

(54) PERSONALIZED CONTEXTUALIZATION OF PATIENT TRAJECTORY

(71) Applicant: The Regents of the University of California, Oakland, CA (US)

(72) Inventors: Pierre-Antoine Gourraud, NANTES (FR); Antoine Lizée, Paris (FR); Stephen L. Hauser, San Francisco, CA (US)

(73) Assignee: The Regents of the University of California, Oakland, CA (US)

(21) Appl. No.: 16/311,122

(22) PCT Filed: Jun. 22, 2017
(86) PCT No.: PCT/US2017/038866

§ 371 Date: Dec. 18, 2018

Related U.S. Application Data
(60) Provisional application No. 62/353,438, filed on Jun. 22, 2016.

(51) Int. Cl. G16H 10/60; G16H 50/70; G16H 50/30; G16H 15/00

(52) U.S. Cl. CPC G16H 10/60; G16H 15/00; G16H 50/30; G16H 50/70

(57) ABSTRACT

Methods, apparatuses, and systems are provided for creating and using a database to identify reference patient data to compare to a current patient. The data can be stored per patient visit, where such a measurement record can store information about the patient, such as a measurement value for a severity of a disease. Data for each patient visit can be accessed separately. A user interface for accessing the database can be configured in a user-friendly manner to allow a user to specify one or more filtering criteria. For example, a user can specify a filtering criterion for reference data to be displayed for a given measurement value of a disease at a particular time (e.g., duration of a disease), and have other filter criteria automatically generated to be applied to reference data at other times.

### CROSS-REFERENCES TO RELATED APPLICATIONS

The present application claims priority from and is a PCT application of U.S. Provisional Application No. 62/353,438, filed Jun. 22, 2016, the entire contents of which are herein incorporated by reference for all purposes.

### STATEMENT AS TO RIGHTS TO INVENTIONS MADE UNDER FEDERALLY SPONSORED RESEARCH AND DEVELOPMENT

This invention was made with government support under grant nos. R01 NS049477, TR000004, and TR000143 awarded by the National Institutes of Health. The government has certain rights in the inventions. The government has certain rights in the invention.

### BACKGROUND

Measured properties of a subject (e.g., a patient with a disease) is often measured and compared against a population. For example, a baby's weight can be measured and compared to other babies having the same age. The comparison can involve a growth chart segmented by percentiles, so that one can determine which percentile the baby's weight corresponds to relative to others. Typical database systems can access such static population data and provide plots easily, permitting the user to track a baby's weight over time.

However, more complex data sets create challenges for existing database systems and interactive user interfaces, particularly for subjects with complex clinical disease states. Here, patients typically have many data elements that must be brought together to create a coherent picture of the dynamic and multifaceted clinical problem. It is difficult to identify, access, and display such data in an efficient and coherent fashion, as well as to display suitable aggregated data from other similarly affected individuals that can be useful for comparison to an individual, current, patient. Such problems are compounded in the era of big data, where patient information is gathered from multiple sources, thereby making it even more difficult for large amount of data in a database to be accessed and analyzed in an efficient, flexible, and user-friendly manner.

Embodiments provide solutions to these and other problems.

### BRIEF SUMMARY

Embodiments provide methods, apparatuses, and systems for creating and using a database to identify data from a reference patient population to compare to a current patient. The database can be configured in a manner to provide efficient identification of the medically relevant data, as well as data useful for research purposes. For example, the data can be stored per patient visit, where such a measurement record can store information about the patient, (e.g., a measurement value for a severity of a disease). Such a separation of patient data into separate measurement records can allow data for each patient visit to be accessed separately, e.g., to accommodate application of different filter criteria for data related to different patient visits.

A user interface for accessing the database can be configured in a user-friendly manner to allow a user to specify one or more filtering criteria. For example, a user can specify a filtering criterion for reference data to be displayed for a given measurement value of a disease at a particular time (e.g., duration of a disease), and have other filter criteria be automatically generated to be applied to reference data at other times. In this manner, the user interface in combination with a back-end analysis can provide a simple mechanism for a user to access the desired information for display.

Other embodiments are directed to systems and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

FIG. 1A shows a plot illustrating a trajectory of a patient in the context of a reference distribution. FIG. 1B shows a plot illustrating trajectory in a different context of a reference distribution.

FIG. 2 shows a diagram illustrating a process for organizing a database and filtering the database based on a user search according to embodiments of the present invention.

FIG. 3 shows a block diagram of a system for creating a database of measurement records and for accessing the database using filters.

FIG. 4 shows different reference patient records (orange) and a current patent record (blue).

FIG. 5 shows the different measurement records being assigned to different subsets

FIG. 6 shows the subsets being used to determined values of the distribution at each time value.

FIG. 7 shows a diagram illustrating some of the subsets being filtered out (the ones with empty blocks).

FIG. 8 shows different categories of data values for measurement records.

FIG. 9A shows the quantiles for the value of 3-6 at year 18, specifically 22^{nd} and 89^{th} percentiles. FIG. 9B shows EDSS values corresponding to those percentiles.

FIG. 10 is a flowchart of a method 1000 of accessing and using a database of patient data according to embodiments of the present invention.

FIG. 11 shows a block diagram of an example computer system usable with system and methods according to embodiments of the present invention.

### DETAILED DESCRIPTION

Although contextualization has long been used for viewing data in a longitudinal format (e.g., growth charts for babies), database and user interfaces have not evolved to handle the new era of big data that includes large amounts of patient data, with many variables and types of information. For example, current techniques only provide ways to access different populations using criteria of immutable characteristics (e.g., sex, race, ethnicity, etc.), but do not provide a good mechanism for contextualization of characteristics that change over time. The data in current databases are not structured in a manner to allow access (e.g., filtering) in a manner that allows time-varying characteristics to easily be used as criteria for accessing the data, and thus are limited in the ability to provide contextualization.

Embodiments can provide database systems and interfaces that allow a user to find patients who are similar to a current patient and that align these data, thereby allowing a user to understand the current patient in the context of many referenced patients with similar characteristics. The data can be stored for efficient searching, and filter criteria can be generated based on simple input from a user, e.g., input about a single time point, where the database system automatically generates filter criteria for other time points.

Accordingly, embodiments can provide a software engine pipeline that enables accurate and robust computing of population aggregated information tailored to the metrics for one patient. Systems can perform such operations dynamically and in real time, so the user can further tailor the contextualization to the patient based on his/her knowledge about the patient. Examples describe use for multiple sclerosis (MS) patients, but one will appreciate that embodiments can be used for other diseases.

Embodiments can address the need of normality assessment for complex medical conditions that present high- and multi-component variability between patients and over time. As a result, embodiments of the software platform can help a person to efficiently access and view a particular set of data in a proper context, e.g., to make decisions in the clinic about a patient based on quantified evidence obtained from the data of many (other) patients. An output of database systems of embodiments can provide the physician with data-driven insights in real time, through a natural, simple interaction.

Accordingly, a goal is to provide physicians with data-driven insights in real-time, at the point of care, through a natural, actionable interaction. Such interaction is improved with new software techniques for storing, accessing, and filtering the data that increase computational speed and increase availability of access to the data by enabling access via less demanding interaction from a user. Data of patients is under-used, and embodiments allow the data be directly used for versatile decision support, e.g., at point of care in clinics or at the hospital bedside, and also permit users to remotely access the data in a secure fashion. Below, we describe the principles of personalized contextualization of patient trajectory. Then, we present an overview of an implementation and then further details. We also propose variations.

### I. Contextualization

Contextualization can refer to the display of one particular entity's data in relation to a customizable distribution in a reference dataset of many similar entities. Contextualization enables quick, normative evaluation of the entity state and evolution over time. Contextualization can promote systematic display of one particular entity's data in the context of the distribution of the same measure or information for many similar entities in aggregate. Embodiments can provide improvements for creating and using databases for retrieving and viewing such data in an efficient and customizable manner. Embodiments can perform a personalized contextualization of patient trajectory (PCPT) to provide such improvements.

A. Presentation of Trajectory

A “trajectory” refers to a group of data points of the same metric referring to one same entity. For example, all the values of a clinical score measured over time for one patient is a trajectory. Embodiments of PCPT can display around the trajectory of one patient said “under study” aggregated values (i.e., retrieved specifically for that patient), which are computed from a population dataset comprised of many patients with a shared trait, like a similar diagnosis.

Embodiments can enable the physician to identify, easily and more accurately, the current state and trend of an individual patient's condition, and what can be expected with respect to future outcomes. This contextualization can (i) be adjusted to accommodate information known about the patient under care or under study (e.g., an initial personalization), and (ii) be provided in real-time to the clinician, researcher, or patient. A second round of personalization of the contextualization can reside in a real-time adjustment of the displayed information to some characteristics of the patient (gender, age, etc.) and of the disease (sub-type of disease, treatment used, year into the disease, etc.) in order to modify the group of similar entities used to compute the population context.

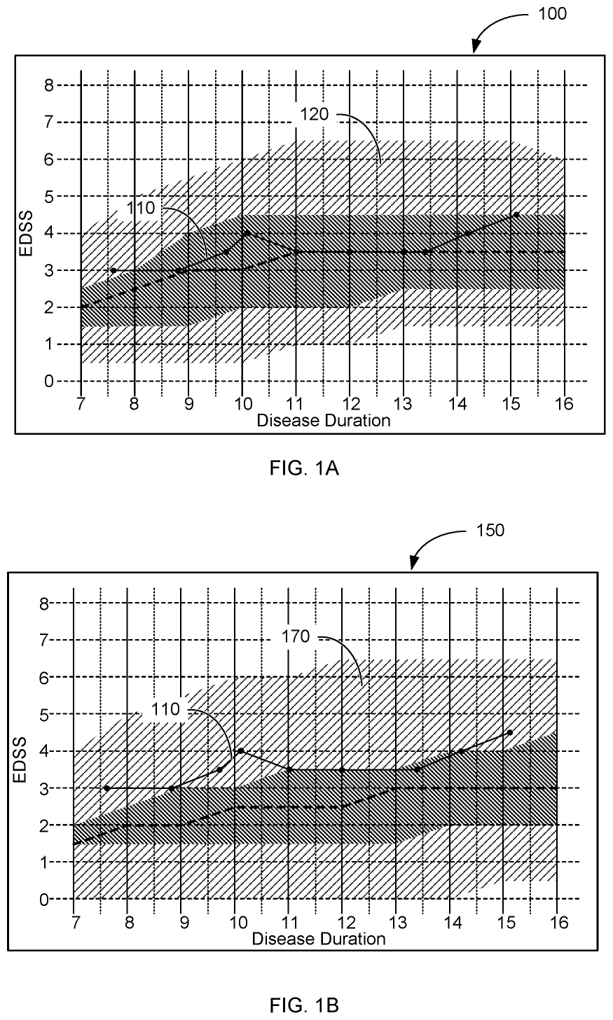

FIG. 1A shows a plot 100 illustrating a trajectory 110 of a patient in the context of a reference distribution 120. The vertical axis (Y-axis) is a measurement value of a disease of a patient. In this example, the patients have multiple sclerosis (MS) and the measurement value is the extended disability status scale (EDSS), which is a functional handicap score developed for multiple sclerosis. Reference distribution 120 shows different percentiles of other patients with respect to the EDSS values.

B. Longitudinal Approach & the Temporal Key

Embodiments can be effective and insightful when used in a longitudinal setting. If the patient data consists of several measures taken at different time points, this data can be laid along a “Temporal Key” (TK) that will define the evolution metric of the trajectory. In various embodiments, this TK can be a direct mapping to the time of the measure (e.g. “disease duration”), but the TK could be any monotonic metric that defines an order relation and a distance between different points of a patient's trajectory. For example, a TK that directly maps to time would be the age of the patient; but one could also use the different stages of the disease to lay out the patient's data in a meaningful manner. This contextualization can occur along the TK, and add “temporal” trending information to the context.

In FIG. 1A, the horizontal axis (X-axis) is disease duration by year. This corresponds to the number of years since the onset of the disease, which can correspond to when the disease is diagnosed or the first symptom or sign is identified. The example in plot 100 compares patients whose disease has the same disease duration at each year. Another example could compare patients based on their age on the X-axis. The X-axis is referred to as a temporal key. The temporal key is a metric that defines the evolution of the patient's trajectory.

In some embodiments, contextualization can be used with “static” trajectories that include only one quantitative point. The contextualization can put in perspective a single value attached to a patient with the distribution of that same metric within a given population. One example would be the intervals that usually assess the normality of lab results.

C. Reference Dataset & Filters

Embodiments can use a “Reference Dataset” (RD). This reference dataset encompasses many individual measurements from a large number of patients, preferably with several points measured at different times for each patient, enabling a longitudinal treatment of the dataset. The RD can enable computation of aggregated statistics. This set of points defining the RD may have many dimensions, e.g., each dimension corresponding to a potential characteristic recorded at each point in time. Structuring the data of the reference dataset allows obtaining the aggregated values characterizing any subset included in the RD. Embodiments can use any selection of dimensions of the RD as “Filters” to create a relevant subset that will be used for computing these aggregated values and thus create the personalized contextualization. To serve this goal, embodiments can structure the data using specific techniques, as described in more detail below.

Reference distribution 120 is computed from a reference data set of reference measurement records. Embodiments provide ways to refine the selection of data points in the reference data set. Various parameters can be used for filter criteria to be used for selecting which data points to include the reference data set for determining reference distribution 120. A simple example is to compare a female patient only to other female patients in the reference dataset. Such a filter criteria is trivial as a reference patient always has a same gender for every visit (thus, gender is an immutable characteristic), while other characteristics of the patient can vary, such as disease severity scores, treatment, and age.

The filtering is more difficult when the criterion involves a characteristic that varies with time. For example, few patients will have only one treatment for the entire duration of a disease, particularly for MS. There may also be a limited period of time in which the patient has been treated with a drug, and thus patients cannot simply be filtered by treatment. To address this problem, embodiments can store measurements records separately, each corresponding to a different visit of a patient. In this manner, only certain measurement records can be selected according to when the patient was receiving a particular treatment (e.g., a particular drug). In this manner, times when the patient is not receiving a particular treatment can be excluded from the reference data. Accordingly, such a data structure of storing and organizing data by patient visit can allow improvement to the functioning of the database.

The reference distribution of data can be used to perform a prognosis of a patient, e.g., a prediction of how the disease will progress. With proper filters entered by a user and implemented by embodiments across the temporal key, a user can increase accuracy in a prognosis.

D. Example of Contextualization

To illustrate the different principles introduced in this section, example results are provided in a real-world setting. FIG. 1B shows a plot 150 illustrating trajectory 110 in a different context of a reference distribution 170. The patient has multiple sclerosis (MS), a life-lasting, autoimmune, neurodegenerative disease with no known cure, but more than 10 known disease-modifying treatment options. The reference dataset was collected at University of California at San Francisco (UCSF) with more than 3,000 visits recorded from more than 500 different patients with MS. Disease duration is the Temporal Key. The “Extended Disability Disease Score” (EDSS) is the metric of the trajectory.

FIGS. 1A and 1B show two different personalized contextualizations from the same reference dataset, selecting as filters for both the Gender (=Male) and the age (33<age<58) of the patient, but varying the filter for one of the most differentiating symptom for MS, Ataxia (TRUE for FIG. 1A, FALSE for FIG. 1B). Ataxia is a neurological sign consisting of lack of coordination of muscle movements that includes gait abnormality. In FIG. 1A, the patient trajectory appears more similar to the median trajectory of the reference dataset (group with Ataxia=TRUE). In FIG. 1B, the patient trajectory appears more similar to the third quartile (defining the top or worst 25%) trajectory of the reference dataset (group with Ataxia=FALSE). This example illustrates the importance of personalized contextualization, since the patient state is initially the same in both cases but becomes quite different when tailoring the contextualization to cases with or without Ataxia.

### II. System Overview

There are challenges in implementing a contextualization tool, e.g., challenges of two kinds. A first difficulty is to adapt to the sparse and diverse nature of data at hand, while providing reliable estimates of the aggregated values. A second issue is to enable real-time computing of this contextualizing data, even when adjusting the parameters (“Filters”) that define the contextualizing population on the fly. To overcome both these challenges, some embodiments use retrospective analyses of datasets preparing pre-computed sets of data points (datasets) that enable real-time computations, thereby providing improvement in the efficiency of the operation of the computer. Embodiments have been successfully implemented on a cloud server to serve remotely real-world applications at the point of care.

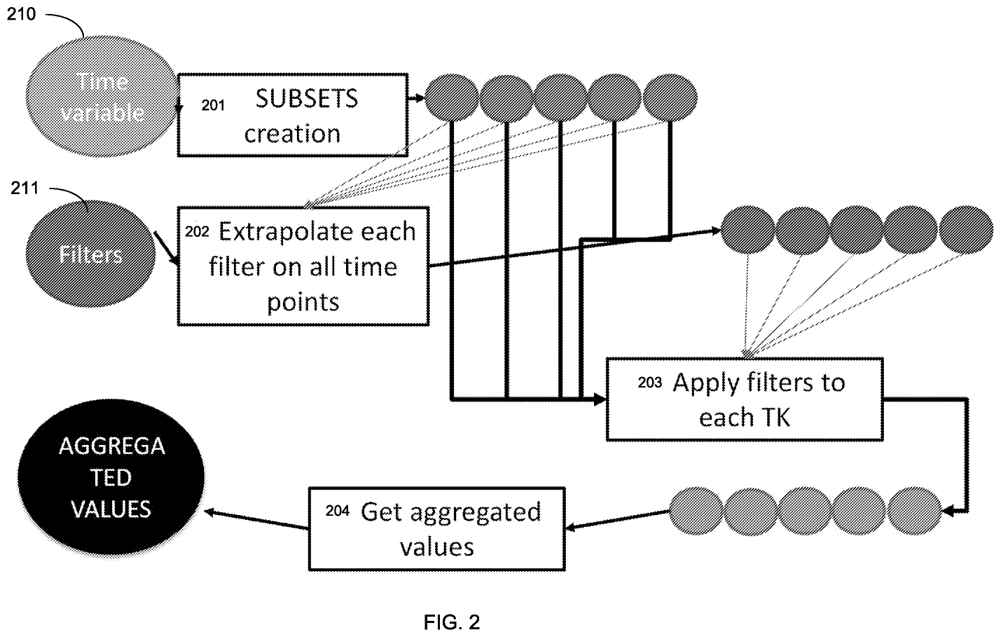

FIG. 2 shows a diagram illustrating a process for organizing a database and filtering the database based on a user search according to embodiments of the present invention. FIG. 2 shows use of a time variable 210 for organizing the database and one or more filters 211 for the filtering.

At block 201, subsets of measurement records are created. A time-related variable (e.g., a temporal key) can be used to segment patient records into separate records for each visit. This can be done by identifying which time value for the time-related variable a particular visit corresponds, e.g., which year of duration that the visit is within. Thus, each subset can correspond to a different value or range of values of the temporal key. This organization of the database allows for a beneficial ability to access particular data in an efficient manner for contextualization.

At block 202, a filter(s) can be obtain from a user for a particular time value (e.g., via any suitable user interface), and the filter(s) for other time values can be determined based on the received filter(s) for the particular time value. As a simple example, if the filter is gender, then the filter can be replicated for applying to all of the subsets. In some implementations, one or more filter criteria for other time values can be extrapolated (or interpolated or fit, e.g., via least squares) from the received filter criteria. For example, an age of a patient for a current disease duration can be extrapolated to determine ages for other time values of the disease duration. A more complicated example is when a particular range for a severity score (e.g., EDSS) is used; this example is discussed in more detail below.

At block 203, each set of filter(s) is applied to the corresponding subset of the corresponding time value, thereby providing filtered subsets. A different filter can be applied to each subset, e.g., when the filter is extrapolated to the other time values. Each time value would have a different extrapolated value for a data characteristic (variable). Thus, in this manner, a single user entry of a filter criterion for one time can provide separate filters, each to be applied at respective times. Each filtered subset can result from applying a specific set of one or more filter criteria that were generated from the user input at the particular time value.

At block 204, aggregated values are obtained from the filtered subsets. The aggregated values (e.g., for a disease-related variable, such as a severity score) can be displayed as a distribution (e.g., percentiles) in a plot such as FIG. 1B. Percentile ranges (quantiles) can be determined at each time value using the filtered subset at that time value.

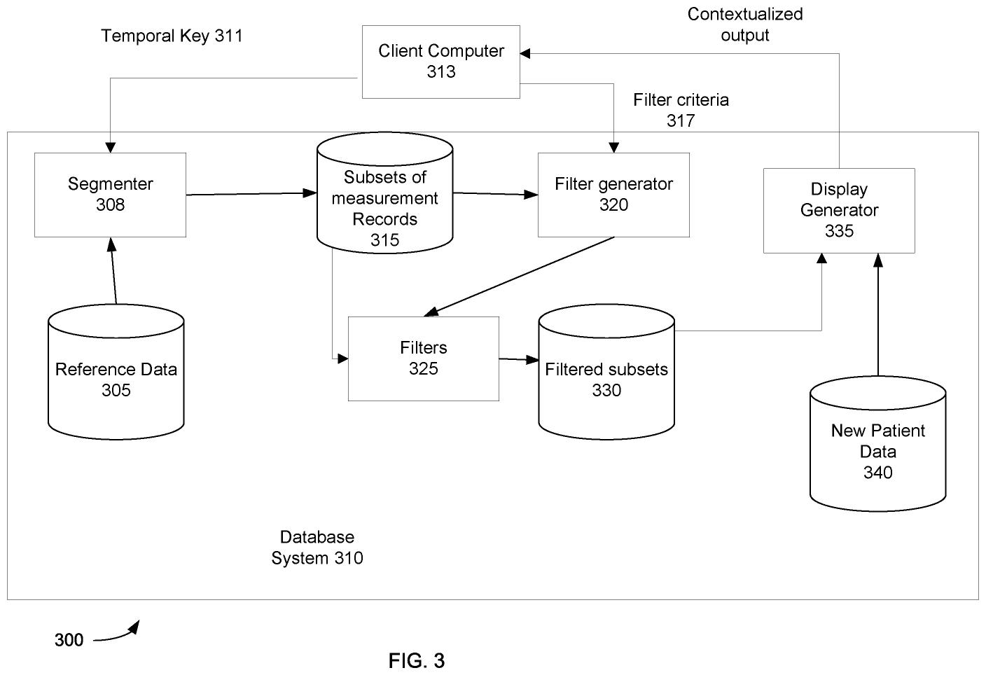

FIG. 3 shows a block diagram of a system 300 for creating a database of measurement records and for accessing the database using filters. System 300 can include reference data 305 that includes patient data that is accessible on a per visit basis. Such accessibility can be provided in a variety of ways, e.g., each record can be a different visit. If needed, a patient identifier can associate different visits to a same patient, but such an identifier is not necessary, as embodiments can treat each visit separately.

A segmenter 308 can receive a temporal key 311 from a client computer 313 that is interacting with database system 310 using a user interface (e.g., a voice interface, a keyboard, a touch screen, etc.). The segmenter 308 can use the temporal key 311 (e.g., disease duration) to segment the reference data 305 into measurement records corresponding to each time value of the temporal key 311. The user can specify the intervals of the time values of the temporal key 311 (e.g., every few years, year, 6 months, etc.). The segmenter 308 can output the subsets of the measurement records.

The client computer 313 can provide one or more filter criteria 317 for a particular time value of the temporal key 311. One or more of the filter criteria 317 can be entered by a user via the user interface and/or can be set at defaults (e.g., via search templates). A filter generator 320 can then generate a set of one or more filters 325 for each subset of measurement records, e.g. by extrapolating the filter criteria 317, which may be provided for just one particular time value. Filter criteria 317 can include filter parameters for more than one time value, and thus interpolation or curve fitting may also be used to determine one or more of the filter parameters of filters 325 that have not been specifically identified. The filters 325 can then be applied to the subsets 315 of measurement records to obtain filtered subsets 330.

A display generator 335 can use the filtered subsets and new patient data 340 to provide the contextualized output. For example, display generator 335 can display a trajectory of the new patient data in the context of the filtered subsets 330. Quantiles of the filtered subsets 330 can be identified.

Accordingly, embodiments can provide improvements over the classic static, indicative ranges used by physicians (e.g. growth charts, lab “normality” range, etc.). One improvement is providing a dynamic contextualization based on the information about the patient (age, gender, etc.), including a time-dependent variable. Another improvement is that, by directly using as input any dataset of consistent individual patient's data, embodiments are applicable to any condition for which such a dataset exists, and thus brings the normality assessment to new fields in medicine. Embodiments can provide decision support in the case of complex diseases characterized by a large between-patient and over-time variability. Such contextualization can deliver a normality assessment of individual data to the clinician, researcher, or patient in order to guide his/her decision, while remaining easy to understand and interpret.

### III. Subsets for Time Values of the Temporal Key

As described above, the dataset can be discretized along a Temporal Key (TK) of choice. The aggregated values can be computed for these values of TK on the points that fall in the subsets that are created. For example, one could discretize the TK “disease duration” by years. All the points closer to year “17” than year “16” or “18”, will create the subset used to compute the aggregated values at year “17” of disease duration.

The subsets of measurement records can be created and stored as a single set of measurement records (e.g., one table per subset). Which subset a measurement record corresponds can be determined based on the time corresponding to the measurement record. Such structuring of the patient data can provide efficient access to the database. Before the creation of the subsets, the gathering of patient records is first discussed.

A. Gathering Data

The data for patient records from various sources can be gathered to be in one database. Usually the data are separated by data type to be in separate databases for genetic, for imaging, for drugs, etc. Embodiments can use a research database that contains information from hospital admissions, outpatient information from clinics, from remotely obtained patient data via telephone, tracking devices, and other sources, from treatment sources, from genomics and other biomarkers including imaging, and from all other types of studies of experiments performed on the patients. The database may exist on a network, referred to as a cloud database. Examples of data include MRI data and genetics data, e.g., from which a summary score can be determined.

The database can be accessed via an API using a user interface that uses the API to provide commands and other data to the database. Some embodiments can use MongoDB, which is a non-relational document database that can ingest data of arbitrary structure and size. A wrapper API can be used to integrate data from biomarker vendors (e.g., whole genome, microarray, serological, etc.), integrate data from electronic medical records, and integrate data from patient-reported tracking devices like FitBit, Nike Plus, heart rate monitors, etc.

Security can be maintained in various ways. For example, MRI data can be acquired and uploaded for preprocessing and analysis. A secure authentication service (token vending machine) can be hosted on the network machines running the database. Authenticated users can receive temporary authorization tokens used to gain access to downstream services. Imaging data can be uploaded to a digital imaging and communications in medicine (DICOM) image service. During a clinical consultation, a clinician can authenticate to the cloud database, retrieve patient images, and if necessary can dynamically analyze patient data relative to a dynamically defined reference population. Data and network communication can be encrypted, images can be skull stripped, and personal information can be removed prior to uploading to cloud to ensure data privacy and HIPAA compliance. Personal health information (PHI) can be processed, stored, or transmitted on PHI approved services. Non-PHI data can use others services

B. Determining Subsets

As described above, visits can be tracked independently for a same patient and different patients.

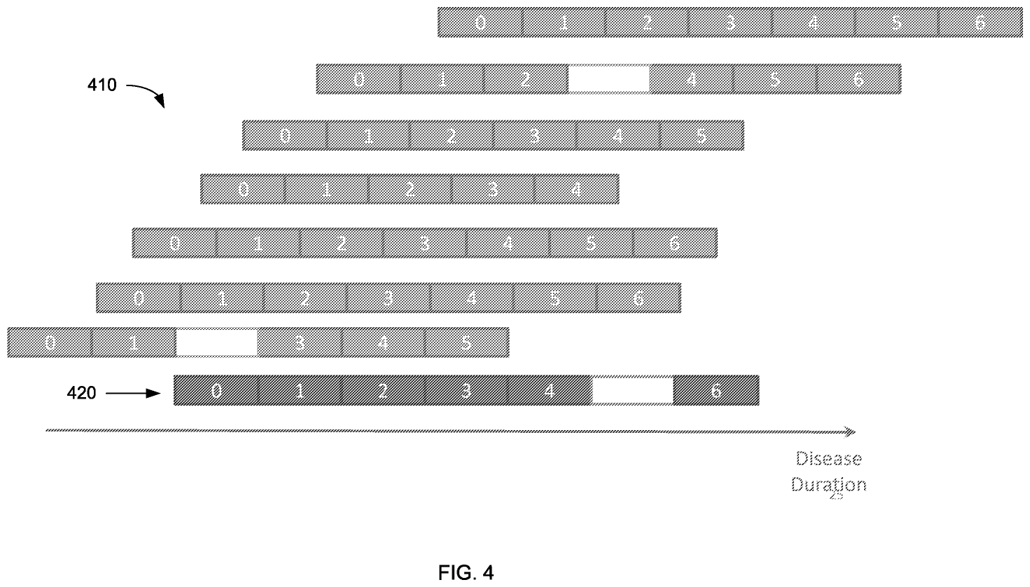

FIG. 4 shows different reference patient records 410 (orange) and the current patent records 420 (blue). The numbers in individual blocks denote an amount of disease duration (e.g., year of disease duration). A purpose of determining the subsets is to contextualize the current patient trajectory.

Some of the reference data set have missing data points, e.g., if a patient missed a visit. As the data in the database may come from various sources, the information for a patient may be very sparse, i.e., not every time value for the temporal key may be available. For example, a first data point of a patient may be after 10 years of the onset of the disease.

A problem is how to align the reference dataset with the current patient. As part of an alignment, the reference data set can be broken into subsets. Each subset has data for different patients, but corresponding to a same time value of the temporal key. Each subset can summarize all the information about what it is to have a disease similar to that of the current patient (blue) at year 12, 13, 14, etc. To determine which subset a measurement record belongs, a time of the measurement record can be used. Then, the time can be compared to ranges for the temporal key, e.g., a range of 1-2, 2-3 year, etc.

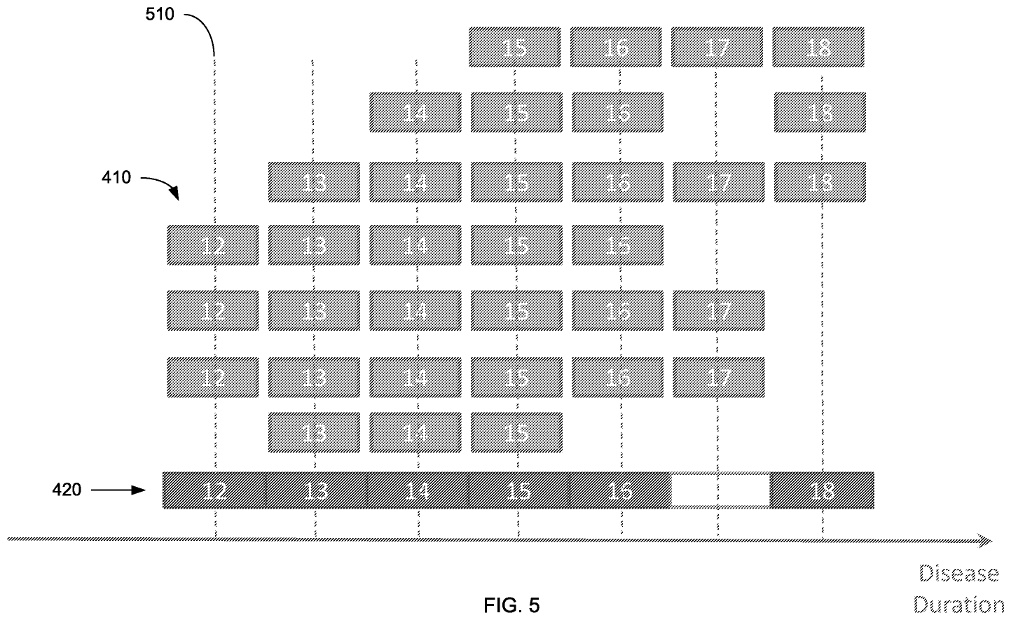

FIG. 5 shows the different measurement records being assigned to different subsets. This process is shown as an alignment of the measurement records that all correspond to a same time value of the temporal key. The different reference patient records 410 (orange) and the current patent records 420 (blue) are shown aligned. Dotted line 510 show the alignment of three reference patient records to the current patient records 420 at disease duration of 12 years.

Once the subsets are determined, properties of the subsets can be determined. For example, a severity score of the disease can be analyzed to determine various values corresponding to different percentiles. The values of the severity score (example of a disease-related variable) can be determined that correspond to the twenty-fifth, fiftieth and seventieth percentiles.

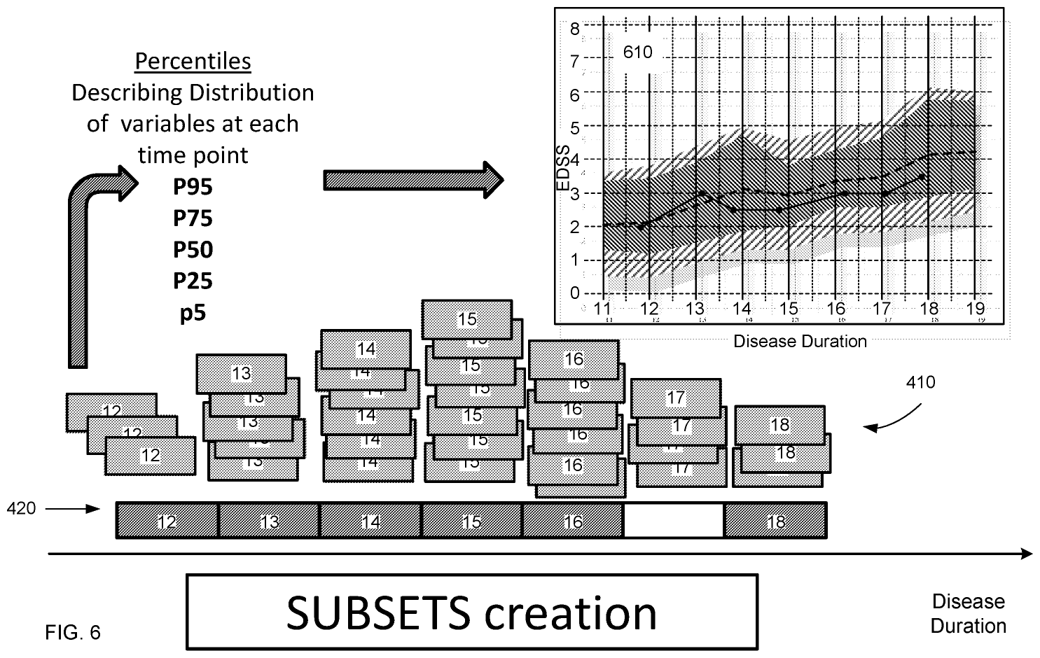

FIG. 6 shows the subsets being used to determined values of the distribution at each time value. In this example, the percentiles of a particular time-varying metric can be determined at 5% (P5), 25% (P25), 50% (P50), 75% (P75), and 95% (P95). These percentiles can then be displayed with the values from the aligned and filtered subsets. Plot 610 shows such an example of the distribution of the filtered subsets being displayed with the trajectory of the current patient.

### IV. Filtering

Personalization of the contextualization to the patient under study can be done through application of filters that define the contextualization population. These filters can be set by the clinician for better reliability, but these can also be preset or auto-adapted. Relying on the physician's insights concerning the disease at stake and the patient under study promotes adoption and can provide a balance between accurate contextualization (many filters) and precise contextualization (many points—fewer filters).

Filtering can be a complex operation that excludes data points of an individual entity contributing to the reference group (e.g. data points after change in prescription for a specific drug treatment). Filtering can also discard all the data of an individual entity contributing to the reference group (e.g., when males are excluded from the analysis).

In some embodiments, for every dimension of data for a patient, embodiments can propose relevant filter parameters. For example, if the patient is age 46, an interval that respects the distribution of ages in the reference dataset can be proposed around this age.

Filtering can be used to select the proper context for analyzing the current patient, i.e., the proper set of reference data. For example, a filter can be to only use patients that have received treatment A. With the use of subsets that have been separated by time value and patient records stored per visit, it may not be known (nor is it necessary to know) whether a given patient has had treatment A at multiple times because the database can actually store information pertaining to whether a patient at a given time point has received a certain treatment. Thus, the filtering process can be viewed as a tool to remove specific measurement records from further analysis.

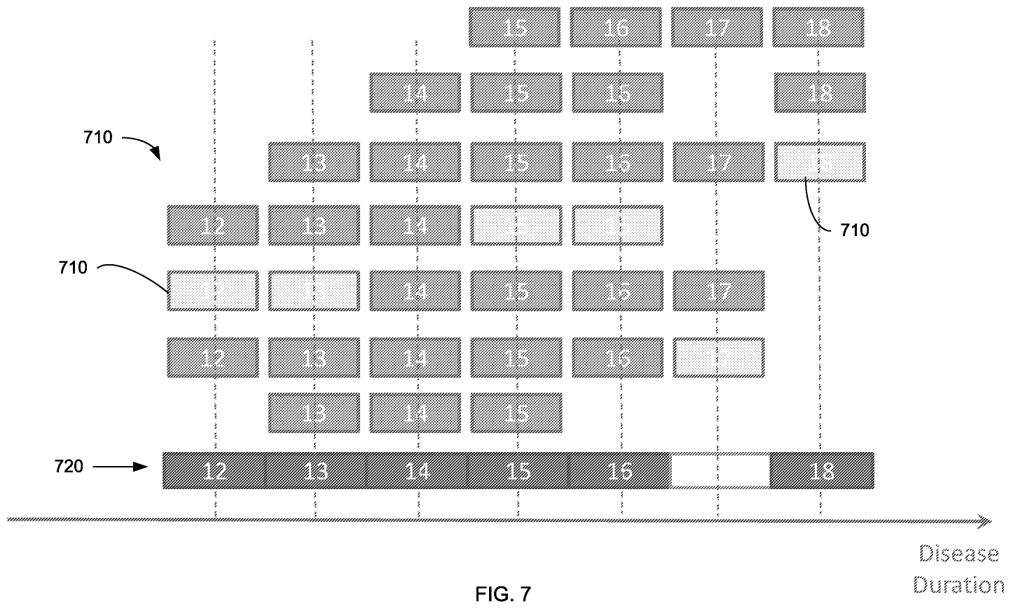

FIG. 7 shows a diagram illustrating some of the subsets being filtered out (the ones with empty blocks). Different reference measurement records 710 (orange) are shown with a current patient record 720 (blue). Reference measurement records 710 have been filtered out, and thus are shown as empty blocks. These reference measurement records 710 do not satisfy the filter criteria, e.g., as generated (as may occur using extrapolation) by filter generator 320. If each row is a single patient, it can be seen that only some of the measurement records for a patient are removed. The discarded records can correspond to years where a patient received a particular treatment that the user has specified for filtering out. In some embodiments, some data points can be added back in, if there is not enough data to provide a statistically accurate analysis.

A. Categories of Filter

In various embodiments, different values of a measurement record can be of different categories. For example, some data values do not change, e.g., gender, whereas others, e.g. treatment, will change.

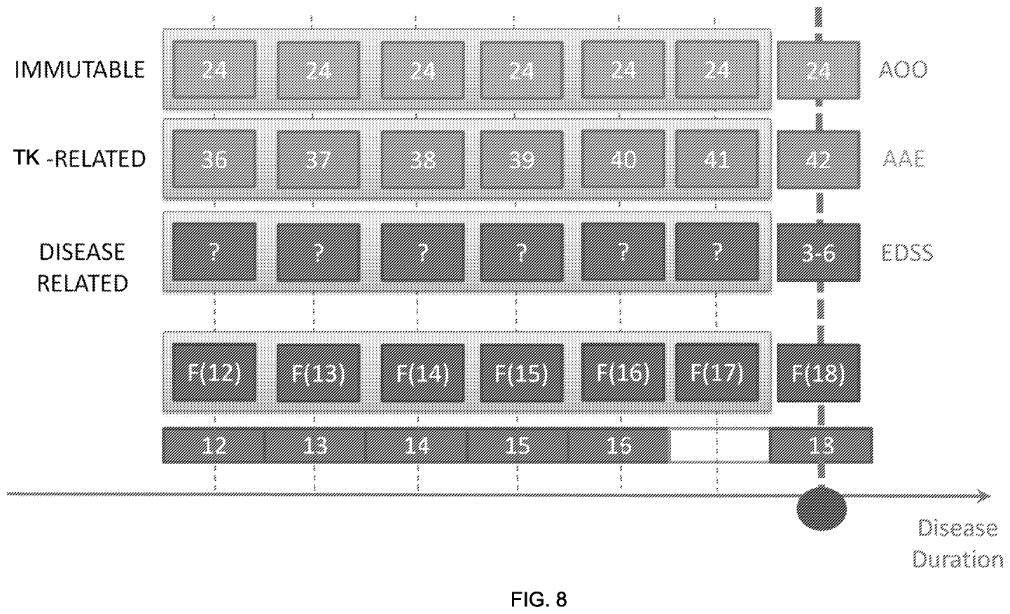

FIG. 8 shows different categories of data values for measurement records. FIG. 8 shows the time value for year 18 being selected by a user. The user then provides filter criteria for year 18. In this example, the user provides a value for the age of onset (AOO), a value for the age at examination (AEE), and a value range for a severity score (EDSS in this example). Specifically, the user provides 24 for AOO, 42 for age at examination, and 3-6 for EDSS.

The age of onset does not change for a patient over time, and thus is considered immutable for a patient. Thus, the value of 24 can be applied to all measurement records regardless of what time value corresponds to the measurement record. Immutable values do not change over time, e.g., biological gender, offset of the disease, etc.

The age of examination does change for different years for the disease duration. Thus, the filter criterion of 42 can be extrapolated to other years for the disease duration. After extrapolation, new filter criteria can be generated at each disease duration. Thus, the filter criterion at year 18 of 42 for AAE gets translated to 41 for disease duration of 17, to 40 at disease duration 16, etc. A range of ages can also be provided in various embodiments. TK-related variables can have a defined relationship to TK, e.g., such that linear (or pseudo-linear, e.g., close to linear) extrapolation regarding the TK can be used, with examples of TK=Disease duration or Filter=Age (e.g., Age+1=disease Duration+1). Other defined relationships can be used, e.g., quadratic or exponential.

Disease-related variables are susceptible to change during the course of disease in a non-predictable way, e.g. clinical scores. The example of the severity score is discussed further below. Since disease-related variables also change over time, a proper contextualization would not simply have an EDSS of 3-6 for the other time values of the disease duration.

By extrapolating the filter criteria entered for the disease direction, one can identify measurements of other patients that are and were similar to the current patient. One can also search for patients that looked similar to a current patient at year 12, and not at the current year 18. This can help to build a predictive model, which can select patients who were like a patient at a previous time, and that data can be used as a training set for prediction.

B. Extrapolation of Filters

The filters may be best applied at one point in time, reflecting the state of the patient's disease at this point—e.g. “now”. Such generation of filters at different times can involve extrapolating the filters to reflect the changes that occur to that variable when the TK varies, e.g., in order to be able to filter the contextualization on the whole timespan of the trajectory in a relevant way. Extrapolation of Immutable or TK-related filters can be done as follows: Immutable filters are TK-independent, thus kept as is and TK-related filters are incrementally modified to reflect changes along the TK. Such extrapolation can use linear or pseudo-linear modelling. But, it becomes complicated for the disease-related variables.

Accordingly, after one or more filter criteria are provided at one point in time, the provided filter criteria can be extrapolated to cover the timespan of the trajectory under study. Filter(s) can be defined for each time point, which are applied separately on the points in each subset created by the discretization of the TK. These subsets of filtered points can then be used for the extraction of the contextualization. For instance, if we are interested in a subpopulation of contextualization that presents physical symptoms of decreased mobility, a filter on Ataxia=TRUE can be set. This filter applies to the points in each subset, not directly to the patients of the reference dataset. In one example, points are “visits” that gather many characteristics for one patient at one timestamp. The filters apply to the visits: as a result, some visits of the patient may be included in the contextualization while others are not. Such exclusion of some visits is a new technique not previously used for the computation of growth curve where the reference data set is fixed, non-pathologic, and comprehensive.

A goal is to reflect the population trend, independently of the contextualized patient's trajectory. Using the one patient “under study” only serves as a starting point for the selection of similar entities in the reference database, and may not influence the modeling of the reference distribution. For instance, some embodiments do not use filter values observed in past data points from the “under study” patient as a basis for the extrapolation, since that can result in a contextualization that artificially centers on the patient trajectory, whereas embodiments can obtain an objective contextualization.

As in FIG. 8, one of the filter criteria can be a range of severity scores, when, e.g., visualizing the evolution of a brain-imaging outcome. For example, one may want to select patients that are plus or minus one point compared to a current patient. If the current patient is 4, that would give a range of 3 to 5. A problem that has not been previously solved is how to propagate that selection back in time.

Some embodiments can translate the percentile of the range, and the range can be propagated back in time maintaining an equivalent percentile. For example, if 3 corresponds to the 5^{th} percentile, then each of the subsets can be analyzed to determine which EDSS values corresponds to the 5^{th} percentile for that subset. As the severity tends to increase with age, the back propagation results in values that decrease. The same can be done for the EDSS values of 6, which can correspond to a 70^{th} percentile, and the EDSS filter values can be determined for the other time values of 12-17 duration years. In this manner, embodiments can automatically determine the filter criteria to be applied to other subsets, e.g., those in the past, or future in other examples.

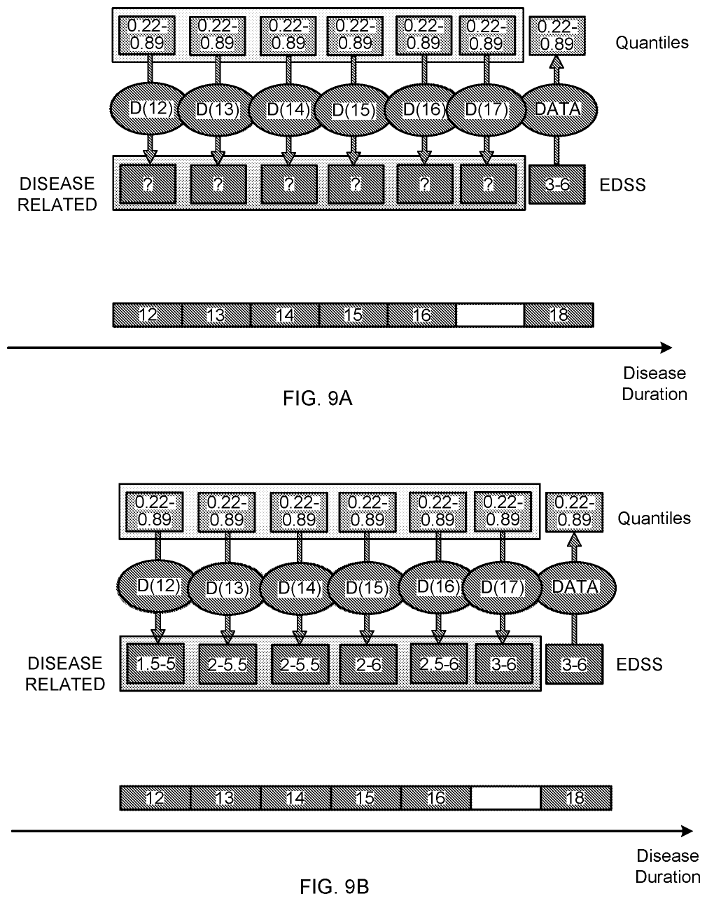

Accordingly, embodiments can use the data of the reference dataset to serve as a basis for the extrapolation process. Some embodiments can proceed by: computing the percentile range (e.g., quantiles) corresponding to the filter applied in the subset at time t (e.g., 13%: 4 EDSS at time t). Then, the values for every other subset at time t−x (e.g. 13% value 3.5 EDSS at time t−1; 3.5 EDSS at t−2, 13%: 3 EDSS at t−3 etc.) can be obtained by applying the same percentile ranges (e.g., 13%) to all the other subsets in order to get the filter bounds, as shown in FIGS. 9A and 9B below.

There are potential issues when doing this data-driven extrapolation, because no value of the measure available at a certain time t−x might equate to the percentile ranges computed for the filter at time t. Embodiments can use different strategies of extrapolation based on the type of filtered disease-related metric, e.g., variable-dependent strategies.

For continuous or discrete variables (e.g.: timed scores, EDSS), embodiments can use the values at time t−x that correspond to the closest percentile ranges to the computed ones, with the additional constraint that the interpolated filter must not shrink the distribution by more than a pre-set shrinking parameter (e.g., equal to 0.7). If it does, the filter is expanded to the value at t−x which corresponds to the next closest percentile range (e.g., quantile).

For binary variables (e.g., Ataxia), if the proportion of the selected outcome is smaller at t−x, embodiments can use the resampling of the two outcomes that minimizes the difference between the expected distribution and target distribution (where both distributions can form a bi-modal distribution of “TRUE” and “FALSE” values) created by the filter at t. If the proportion is larger, embodiments can use the same binary filter. For instance, if “Ataxia=True” is selected at a time point where 30% of the patients have Ataxia, two cases can arise when embodiments extrapolate this filter at a different time point. In a first case, 20% of the patients have ataxia for the different time point, in which case the extrapolated filter will be selecting All ‘Ataxia=True’ patients and a small proportion (12.5%) of the ‘Ataxia=False’ patients, so that 30% of patients are finally selected in total. In a second case, 40% of patients have ataxia at a different time point, and the extrapolated filter by embodiments will be the same, i.e. selecting all (and only) the patients with ‘Ataxia=True’.

For categorical variables (e.g. disease course), embodiments can recode each category filter as a binary variable for the purpose of the extrapolation. Each category can be considered as a separate binary variable for this extrapolation.

FIG. 9A shows the quantiles for the value of 3-6 at year 18, specifically 22^{nd} and 89^{th} percentiles. The data bubbles correspond to the measurement records for the duration years. FIG. 9A can correspond to the user input provided for the particular TK value of 18, which was translated to particular quantile values 22% to 89%.

FIG. 9B shows EDSS values corresponding to those percentiles. These ranges of EDSS values can be used to filter out measurement records that do not correspond to those ranges. Then, when the contextualization is done, only the EDSS values in those ranges at each duration year will be used for the reference data. As shown in this example, the quantiles stay the same but correspond to different EDSS values at different TK values.

C. Missing Data

Handling missing data when filtering can pose considerable challenges. For example, if the data field that is to be filtered is missing, it can be determined whether the patient data for that visit should automatically be excluded. In various embodiments, missing data can be handled in two ways: (i) during the extrapolation of filters for disease-related variables, whether to include the data of a patient visit in the distribution of values on which to compute the extrapolated values, and (ii) during the application of the filters themselves to decide which data should be kept. In one implementation, a mixed approach is used, i.e., using both strategies.

As one example, filtering can be precluded with a variable that has more than 50% missing data in the reference data (RD) as a whole. As another example, imputed values can be used for extrapolation of disease-related filters, as described in more detail below. In some implementations, only real values (e.g., numerical variables) can be used to exclude visits that do not fall into the extrapolated filters.

### V. Extraction of Context (Aggregated Values)

Once each subset has been filtered accordingly, a set of aggregated values can be computed on these subsets for defining the contextualization. For example, once the filtered subsets are determined, the remaining measurement records for each subset can be used to determine any statistical values for the distribution of those remaining measurement records. Various implementations can use the 5^{th}, 25^{th}, 75^{th}, 95^{th} percentiles as well as a measure of the robust mean.

In some embodiments, additional steps can be added to this extraction of the contextualization, e.g., to fill in missing data. For example, before computing the aggregated values, a “longitudinal regularization” can be performed in which the visits of one patient selected in subset t can be propagated into the visits of subset t+1. This procedure could be written as: “For any subset at t, consider any patient who has a visit in this subset. If the next visit of the patient corresponds to kt=t+1 and is not in the filtered subset at t+1, add the visit.” In one aspect, this procedure can be done only once per subset, before any addition of the visits from the previous one. Therefore, in some embodiments, a patient can propagate only one subset forward.

For optimization of the population structure for enrichment, embodiments can optimize the determination of subsets for any possible range of TK, for each meaningful choice of TK. For example, a gradient descent algorithm can be used that translates along the TK trajectories of some patients within the reference dataset to determine an intervening value for a missing field in order to mitigate the sparsity of the data.

For imputation of missing values for filtering, embodiments can use a k-nearest neighbors method for fast imputation (e.g., more than one nearest neighbor). Implementations can use various methods (e.g., a recursive Bayesian approach, interpolation, or curve fitting) for more accuracy in how determining the value that is propagated forward or backward. Accordingly, the integration of longitudinal information to fill in missing data can smooth the data. Similar techniques can also be used to smooth existing data.

After any longitudinal regularization and the computation of the aggregate values, embodiments can further smooth each of the trajectories created by these aggregated values across the range of TK being considered. Embodiments can adjust for the variability in these trajectories that stems from the sparsity of the data. A locally weighted scatterplot smoothing (Loess) interpolation may be used, which can be slightly modified. Accordingly, in some embodiments, the disease values at the percentiles can be smoothed.

### VI. Alternative Implementations

Embodiments can provide several advantages, such as being fast, reliable and transparent. Indeed, because of the structure of the software, the software can be computationally efficient, easy to improve and debug, and easy to interpret. Further issues can be addressed by alternative implementations.

Less-structured datasets may run slower. When different variables are measured at different points in time—instead of being grouped by “visits”—the subset and filtering approach can decrease in precision. Regarding adaptation to data density, if less data is available for certain ranges of the TK, the resolution can remain the same (based on the discretization of the TK). The subset approach can lead to poor resolution along the TK. Additionally, filtering may not implement any interaction between filters as the reverse look-up and application of the filters can be done separately.

A. Sliding Window Implementation Variants

Several discrete steps of the determining subsets could be implemented by a sliding window approach followed by a smoothing algorithm. This can be equivalent to using overlapping subsets as opposed to non-overlapping subsets. For example, after having filtered in or out the visits, one can directly use the position of the visits along the TK to compute sliding versions of the aggregated values, which can correspond to using subsets that share some patient data (i.e., overlapping data between subsets). Thus, subsets are not restricted to having mutually exclusive data.

During the filtering extrapolation step, however, computing the quantiles corresponding to the values of a disease-related filter at a certain point in time has no easy solution—one needs to take an arbitrary number of neighboring points to create the dataset used to compute the quantiles. Then, the same technique of sliding window quantile computation with smoothing algorithm could provide the interpolated filter values for any value of the TK, to be applied in selecting the visits.

But, the problem of sparse structure may remain, e.g., few data points may exist at a particular time. If variables used for filtering are measured at a different time than the variable of interest in the trajectory, one should devise a selection rule that links the variable under-study with the filtering values for each patient—e.g. “closest neighbor”: the closest measure (in “TK-distance”) of the filter variable (e.g., Ataxia) will determine the inclusion/exclusion of each measure of the variable at stake (e.g., EDSS).

B. Model-Based

The evolution of the distribution of any variable Y along the TK could be modeled based on the reference data. This modeled distribution could be used during reverse look-ups in filter extrapolations, and the model re-evaluated for the filtered value. In some embodiments, the process can be as follows: the general models for any Y=f(TK) are computed off-line; then these models are used, in conjunction with some selection rule for sparse data, to filter in/out the measure of the variable under study; the model is re-fitted on the remaining values.

This approach is promising, also because well implemented it could lead to many optimizations and run at least as fast as the current one. The model can be parametric to model a distribution at every point in TK. Also, the model estimates should not depend on the amount of data filtered.

### VII. Method

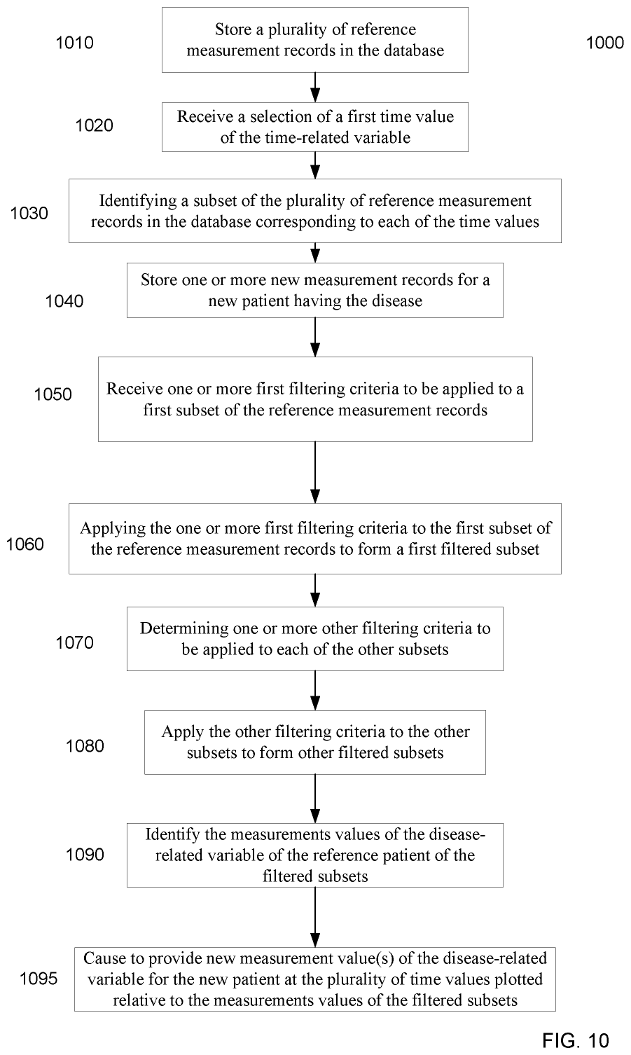

FIG. 10 is a flowchart of a method 1000 of accessing and using a database of patient data according to embodiments of the present invention. The method can be performed by one or more processors communicably coupled with the database. The one or more processors can reside on a server that is communicably coupled with the database and with a client computer on which a user interface resides. In some embodiments, the computer system can include a server computer and a client computer.

Block 1010 stores a plurality of reference measurement records in the database. Each reference measurement record can include: a measurement value of a disease-related variable of a disease of a reference patient and a time value of a time-related variable associated with when the measurement value of the disease-related variable of the reference patient was obtained. A reference measurement record can further include one or more other data values about the reference patient, such as gender and age. The reference measurement records can correspond to reference data described above and can be obtained from various sources.

In some embodiments, the plurality of reference measurement records can be created by analyzing a plurality of reference patient records, which can be recited by a computer system. Each of the reference patient records can include a set of measurement values of a corresponding patient. Then, for each set of measurement values. It can be determined which of a plurality of time values corresponds to a time when the measurement value was obtained. For example, a predetermined set of time intervals can exist, and it can be determined which time interval a measurement was taken. A reference measurement record can then be created using the determined time value, the measurement value, and any other data of the corresponding patient about the corresponding patient at the time. Such measurements records can thus be stored on a per visit basis.

Block 1020 receives, via a user interface, a selection of a first time value of the time-related variable. For example, a user can enter a time value into a search box. As another example, a user can select a time value from a list (e.g., via a dropdown list). Besides such active entering of time, a user can also select to proceed with a default value, e.g., after the default vale is displayed or otherwise provided to the user. Such a selection of proceeding is still considered to be performed via a user interface.

Block 1030 identifies a subset of the plurality of reference measurement records in the database corresponding to the time value. Block 1030 can be performed for each of a plurality of time values of the time-related variable. In some embodiments, each subset of the plurality of reference measurement records can be stored in a separate table in the database. Thus, the identifying can be performed by identifying tables that correspond to the plurality of time values. The time values for the subsets can be time intervals, and thus not every record of a subset needs to have exactly the same time; they just need to fall in a same time interval. In various embodiments, the plurality of time values can correspond to all time values (e.g., all time intervals specified, as may occur in a directory of the database), a set of default time values, or time values selected by a user.

Block 1040 stores one or more new measurement records for a new patient having the disease. At least one of the new measurement records can correspond to the first time value. For example, a new measurement record may correspond to a particular age or disease duration (or other time value) of the patient, where the particular age or disease duration falls within an interval defined by the first time value. The one or more new measurement records can be stored within a same table, where each row has a field corresponding to a time that a measurement was obtained. In one implementation, the time-related variable is a duration of a disease shared by the reference patients and the new patient.

Block 1050 receives, at the user interface, one or more first filtering criteria to be applied to a first subset of the reference measurement records. The first subset can correspond to the first time value of the time-related variable. For example, a user can specify an age or disease duration, and then provide a filter to be applied to the subset of records at that age or disease duration. The filters may be of various types, e.g., of immutable variables, other time-related variables, or disease-related variables. The first filtering criteria can be provided just for the first subset, with later blocks determining and applying filtering criteria generated from the first filtering criteria.

Block 1060 applies the one or more first filtering criteria to the first subset of the reference measurement records to form a first filtered subset that includes reference measurement records corresponding to the first time value and satisfying the one or more first filtering criteria. For example, the first filtering criteria can specify a gender, and records relating to the non-matching gender can be removed. Other filtering criteria can correspond to ranges of a disease related variable or other examples provided herein. The application of the filtering criteria can be implemented by accessing fields of the subset of records (e.g., accessing a particular field of a table corresponding to the subset) to determine which records match.

Blocks 1070 and 1080 can be performed for each of the each of other subsets of the reference measurement records corresponding to other time values of the time-related variable.

Block 1070 determines one or more other filtering criteria to be applied to the other subset. The one or more other filtering criteria are determined using the one or more first filtering criteria. For example, block 1070 may involve simply copying first filtering criteria (e.g., for immutable characteristics) to the one or more other filtering criteria. As another example, the one or more other filtering criteria can be extrapolated from the one or more first filtering criteria. Such extrapolation is described herein.

Block 1080 applies the one or more other filtering criteria to the other subset to form another filtered subset that includes reference measurement records corresponding to the other time value and satisfying the one or more other filtering criteria. Once the one or more other filtering criteria are determined, they can be applied to a particular subset by accessing the records of the subset, e.g., by accessing a corresponding field to determine whether it matches the one or more other filtering criteria.

In some embodiments, the receiving of the one or more first filtering criteria includes receiving a first range for the disease-related variable at the first time value. For example, a range of 3-6 EDSS can be received, when the disease is MS. When the first filtered subset includes reference measurement records having a measurement values within the first range, the records of the first filtered subset can be analyzed to determine an extrapolation (or other translation, such as interpolation or curve fit) of the one or more first filtering criteria for the disease-related variable to the other time values. In one implementation, a percentile range corresponding to the first range can be determined. The percentile range can be determined relative to the measurement values of the first subset. A second range of the measurement values that correspond to the percentile range for each of the other subsets can be determined. For instance, a percentile range of 25%-75% would correspond to different measurement values for different time values (e.g., different severity at different age or disease duration). Once the one or more other filtering criteria are applied to a particular subset, it can be determined which reference measurement records have a measurement values within the other range corresponding to the other filtered subset.

Block 1090 identifies the measurements values of the disease-related variable of the reference patient of the filtered subset, for each of the filtered subsets of the reference measurement records. The identified measurements values can provide the desired contextualization desired by the user, e.g., corresponding to the first filtering criteria that were only specified for one time value. In this manner, the user can obtain greater flexibility in the contextualization while not increasing the work for the user. That is, the user interface can be simple and allow disease-related variables to be used in a similar manner as immutable values.

Block 1095 provides one or more new measurement values of the disease-related variable for the new patient at the plurality of time values plotted relative to the measurements values of the filtered subsets. Thus, the one or more new measurements values that are known can be compared to a properly normalized reference range. The providing of the one or more new measurement values can include displaying the one or more new measurement values.

Block 1095 may be performed by a client device that receives the measurements values from a server, e.g., one that stores the reference patient data. Thus, the server may cause the client device to provide such a plot. Accordingly, the client device can perform the storing in block 1040 and any display for 1095, while other blocks can be performed by the server with the reference patient data.

In some embodiments, operations described above for filtering can be done in real time. The operations involved are computationally accessible and the whole contextualization for the same range of TK, the same patient, and ˜20 different variables of interest is typically executed in the 10-100 ms range, and would be expected to improve further with computational advances. The datasets can be prepared for the real-time operations. These preparation operations can require more computation time, but do not have to be done each time a contextualization is created.

A personalized medicine support system can be implemented for one patient by comparing the trajectory of one patient to the distribution of trajectories in a referenced set of population of patients. By being able to compare the trajectory of any metric of a patient to a reference population that can be tailored to the specificity of this patient's disease, embodiments can provide an artificial experience to the clinician. As a result, non-experts can use, at the point of care, the most advanced or disease-specific metrics that he/she may not be typically use to incorporate in his/her assessment of the patient's disease evolution. This successfully informs the clinician's decision making at all levels. Additionally, the contextualization provides strong visual cues that can be interpreted by both medical specialists and other experts, as well as by non-experts, which efficiently improves the quality of communication between the clinician and patient and permits patients to more fully participate in his/her care.

In order to serve the user-friendly frontend (e.g., tablet computers), embodiments can isolate the computation of a PCPT (e.g., on a cloud server with a dedicated analytical computation engine), which can provide flexibility and scalability; the Reference Dataset or the implementation of the algorithm can evolve separately, without the need of updating the frontend. This backend system can be hosted on a HIPAA-compliant cloud that presents APIs, and the data is served to the frontend through classic RESTFul HTTP/JSON communication. The refresh of the contextualization can take 100-300 ms depending on the network conditions and queries.

### VIII. Computer System

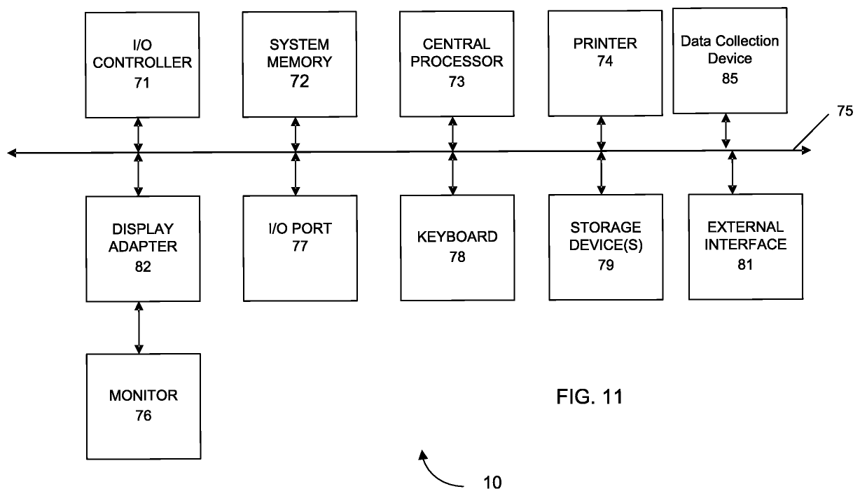

Any of the computer systems mentioned herein may utilize any suitable number of subsystems. Examples of such subsystems are shown in FIG. 11 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 11 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

1. A method of accessing and using a database of patient data, the method comprising performing, by one or more processors communicably coupled with the database:
storing a plurality of reference measurement records in the database, each reference measurement record including:
a measurement value of a disease-related variable of a disease of a reference patient,
a time value of a time-related variable associated with when the measurement value of the disease-related variable of the reference patient was obtained;
receiving, via a user interface, a selection of a first time value of the time-related variable;
for each of a plurality of time values of the time-related variable:
identifying a subset of the plurality of reference measurement records in the database corresponding to the time value;
storing one or more new measurement records for a new patient having the disease, wherein at least one of the one or more new measurement records corresponds to the first time value;
receiving, at the user interface, one or more first filtering criteria to be applied to a first subset of the reference measurement records, the first subset corresponding to the first time value of the time-related variable;
applying the one or more first filtering criteria to the first subset of the reference measurement records to form a first filtered subset that includes reference measurement records corresponding to the first time value and satisfying the one or more first filtering criteria;
for each of other subsets of the reference measurement records corresponding to other time values of the time-related variable:
determining one or more other filtering criteria to be applied to the other subset, wherein the one or more other filtering criteria are determined using the one or more first filtering criteria;
applying the one or more other filtering criteria to the other subset to form another filtered subset that includes reference measurement records corresponding to the other time value and satisfying the one or more other filtering criteria;
for each of the filtered subsets of the reference measurement records:
identifying the measurements values of the disease-related variable of the reference patient of the filtered subset; and
causing a providing of one or more new measurement values of the disease-related variable for the new patient at the plurality of time values of the time-related variable plotted relative to the measurements values of the filtered subsets.

2. The method of claim 1, wherein each subset of the plurality of reference measurement records is stored in a separate table in the database.

3. The method of claim 1, wherein providing the one or more new measurement values includes displaying the one or more new measurement values.

4. The method of claim 1, wherein receiving the one or more first filtering criteria includes receiving a first range for the disease-related variable at the first time value, wherein the first filtered subset includes reference measurement records having a measurement values within the first range;
wherein determining the one or more other filtering criteria includes:
determining a percentile range corresponding to the first range, the percentile range determined relative to the measurement values of the first subset,
determining a second range of the measurement values that correspond to the percentile range for each of the other subsets; and
wherein each of the other filtered subsets include reference measurement records having a measurement values within the other range corresponding to the other filtered subset.

5. The method of claim 1, further comprising:
receiving a plurality of reference patient records, each including a set of measurement values of a corresponding patient;
for each set of measurement values:
determining which of the plurality of time values corresponds to a time when the measurement value was obtained; and
creating a reference measurement record using the determined time value, the measurement value, and any other data of the corresponding patient about the corresponding patient at the time.

6. The method of claim 1, wherein each reference measurement record further includes one or more other data values about the reference patient, and wherein the one or more other data values about the reference patient include at least one of gender and age.

7. The method of claim 1, wherein the time-related variable is a duration of a disease shared by the reference patients and the new patient.

8. The method of claim 1, wherein the disease-related variable is EDSS for multiple sclerosis.

9. A computer product comprising a computer readable medium storing a plurality of instructions for controlling a computer system to perform a method of accessing and using a database of patient data, wherein the method comprises:
storing a plurality of reference measurement records in the database, each reference measurement record including:
a measurement value of a disease-related variable of a disease of a reference patient,
a time value of a time-related variable associated with when the measurement value of the disease-related variable of the reference patient was obtained;
receiving, via a user interface, a selection of a first time value of the time-related variable;
for each of a plurality of time values of the time-related variable:
identifying a subset of the plurality of reference measurement records in the database corresponding to the time value;
storing one or more new measurement records for a new patient having the disease, wherein at least one of the one or more new measurement records corresponds to the first time value;
receiving, at the user interface, one or more first filtering criteria to be applied to a first subset of the reference measurement records, the first subset corresponding to the first time value of the time-related variable;
applying the one or more first filtering criteria to the first subset of the reference measurement records to form a first filtered subset that includes reference measurement records corresponding to the first time value and satisfying the one or more first filtering criteria;
for each of other subsets of the reference measurement records corresponding to other time values of the time-related variable:
determining one or more other filtering criteria to be applied to the other subset, wherein the one or more other filtering criteria are determined using the one or more first filtering criteria;
applying the one or more other filtering criteria to the other subset to form another filtered subset that includes reference measurement records corresponding to the other time value and satisfying the one or more other filtering criteria;
for each of the filtered subsets of the reference measurement records:
identifying the measurements values of the disease-related variable of the reference patient of the filtered subset and
causing a providing of one or more new measurement values of the disease-related variable for the new patient at the plurality of time values of the time-related variable plotted relative to the measurements values of the filtered subsets.

10. A system comprising:
a database of patient data; and
one or more processors communicably coupled with the database and configured to perform:
storing a plurality of reference measurement records in the database, each reference measurement record including:
a measurement value of a disease-related variable of a disease of a reference patient,
a time value of a time-related variable associated with when the measurement value of the disease-related variable of the reference patient was obtained;
receiving, via a user interface, a selection of a first time value of the time-related variable;
for each of a plurality of time values of the time-related variable:
identifying a subset of the plurality of reference measurement records in the database corresponding to the time value;
storing one or more new measurement records for a new patient having the disease, wherein at least one of the one or more new measurement records corresponds to the first time value;
receiving, at the user interface, one or more first filtering criteria to be applied to a first subset of the reference measurement records, the first subset corresponding to the first time value of the time-related variable;
applying the one or more first filtering criteria to the first subset of the reference measurement records to form a first filtered subset that includes reference measurement records corresponding to the first time value and satisfying the one or more first filtering criteria;
for each of other subsets of the reference measurement records corresponding to other time values of the time-related variable:
determining one or more other filtering criteria to be applied to the other subset, wherein the one or more other filtering criteria are determined using the one or more first filtering criteria;
applying the one or more other filtering criteria to the other subset to form another filtered subset that includes reference measurement records corresponding to the other time value and satisfying the one or more other filtering criteria;
for each of the filtered subsets of the reference measurement records:
identifying the measurements values of the disease-related variable of the reference patient of the filtered subset; and
causing a providing of one or more new measurement values of the disease-related variable for the new patient at the plurality of time values of the time-related variable plotted relative to the measurements values of the filtered subsets.

11. The computer product of claim 9, wherein each subset of the plurality of reference measurement records is stored in a separate table in the database.

12. The computer product of claim 9, wherein providing the one or more new measurement values includes displaying the one or more new measurement values.

13. The computer product of claim 9, wherein receiving the one or more first filtering criteria includes receiving a first range for the disease-related variable at the first time value, wherein the first filtered subset includes reference measurement records having a measurement values within the first range;
wherein determining the one or more other filtering criteria includes:
determining a percentile range corresponding to the first range, the percentile range determined relative to the measurement values of the first subset,
determining a second range of the measurement values that correspond to the percentile range for each of the other subsets; and
wherein each of the other filtered subsets include reference measurement records having a measurement values within the other range corresponding to the other filtered subset.

14. The computer product of claim 9, wherein the method further comprises:
receiving a plurality of reference patient records, each including a set of measurement values of a corresponding patient;
for each set of measurement values:
determining which of the plurality of time values corresponds to a time when the measurement value was obtained; and
creating a reference measurement record using the determined time value, the measurement value, and any other data of the corresponding patient about the corresponding patient at the time.

15. The computer product of claim 9, wherein the time-related variable is a duration of a disease shared by the reference patients and the new patient.

16. The system of claim 10, wherein each subset of the plurality of reference measurement records is stored in a separate table in the database.

17. The system of claim 10, wherein providing the one or more new measurement values includes displaying the one or more new measurement values.

18. The system of claim 10, wherein receiving the one or more first filtering criteria includes receiving a first range for the disease-related variable at the first time value, wherein the first filtered subset includes reference measurement records having a measurement values within the first range;
wherein determining the one or more other filtering criteria includes:
determining a percentile range corresponding to the first range, the percentile range determined relative to the measurement values of the first subset,
determining a second range of the measurement values that correspond to the percentile range for each of the other subsets; and
wherein each of the other filtered subsets include reference measurement records having a measurement values within the other range corresponding to the other filtered subset.

19. The system of claim 10, wherein the one or more processors are further configured to perform:
receiving a plurality of reference patient records, each including a set of measurement values of a corresponding patient;
for each set of measurement values:
determining which of the plurality of time values corresponds to a time when the measurement value was obtained; and
creating a reference measurement record using the determined time value, the measurement value, and any other data of the corresponding patient about the corresponding patient at the time.

20. The system of claim 10, wherein the time-related variable is a duration of a disease shared by the reference patients and the new patient.
